(19) United States
(12) Patent Application Publication
(10) Pub. No.: US 2025/0375574 A1
(43) Pub. Date: Dec. 11, 2025

(54) METHOD FOR IMPROVING BLOOD GLUCOSE CONTROL OF A HYBRID CONTROLLER

(71) Applicant: UNIVERSITAT POLITÈCNICA DE VALÈNCIA, Valencia (ES)

(72) Inventors: Iván SALA MIRA, Valencia (ES); Pedro José GARCÍA GIL, Valencia (ES); Jorge BONDÍA COMPANY, Valencia (ES); José Luis DIEZ RUANO, Valencia (ES)

(21) Appl. No.: 19/099,328

(22) PCT Filed: Jun. 12, 2023
(86) PCT No.: PCT/ES2023/070385

§ 371 Date: Jan. 28, 2025

(30) Foreign Application Priority Data
Jul. 28, 2022 (ES) P202230693

(51) Int. Cl. A61M 5/172; G16H 40/63

(52) U.S. Cl. CPC A61M 5/1723; G16H 40/63; A61M 2230/201

(57) ABSTRACT

A method for improving blood glucose control of a hybrid controller meal and exercise announcement by substituting patient-initiated meal boluses of said hybrid controller by an automatic insulin correction signal without retuning of said hybrid controller, and by incorporating rescue carbohydrates suggestion for hypoglycemia mitigation, comprising the steps of measuring a plasma glucose (G(t)) signal by means of a continuous glucose monitor (CGM), calculating a glucose level (Ĝ(t)) by using a glucose-insulin model; computing a disturbance term d(t), generating a virtual signal u_{IMC}(t), for mitigating the effect of d(t) on the output, by means of an IMC filter Q(s) and converting the virtual signal u_{IMC}(t) into three feed forward actions: insulin infusion, rescue carbohydrate suggestion, and insulin-on-board reduction.

### OBJECT OF THE INVENTION

The present invention is framed in the field of medical procedures and devices, and more particularly, in the diabetes care.

An object of the present invention is a method for improving blood glucose control of a hybrid controller allowing to eliminate the need for meal and exercise announcement by substituting patient-initiated meal boluses of said hybrid controller by an automatic insulin correction signal without retuning of said hybrid controller, and by incorporating rescue carbohydrates suggestion for hypoglycemia mitigation.

Another object of the invention is an add-on module for incorporating in a hybrid artificial pancreas, which incorporates a modified Internal Model Control that removes meal and exercise announcements.

Another object of the invention is an artificial pancreas system which includes the add-on module for carrying out the method of the invention.

### BACKGROUND OF THE INVENTION

Closed-loop glucose control, i.e. artificial pancreas, outperforms other insulin therapies treating type 1 diabetes, such as multiple daily injections insulin therapy or sensor-augmented pump.

This technology reduces time in high glucose values (hyperglycemia) and associated risks such as retinopathy, neuropathy, or cardiovascular disease. The artificial pancreas also reduces time in low blood glucose values (hypoglycemia), with critical short-term complications (e.g., cognitive dysfunction, seizures, or coma in severe cases), and enhances the quality of life, by reducing anxiety or insomnia among others.

However, external disturbances, namely, meals and exercise, challenge the performance of artificial pancreas systems.

On the one hand, glucose ingested from meals reaches the bloodstream faster than subcutaneous insulin. Slow subcutaneous insulin absorption and sensor lag delay the insulin action, leading to sizeable postprandial glucose excursions. Insulin stacked in subcutaneous depots continues to be absorbed even after the meal absorption, which may also cause hypoglycemia.

On the other hand, exercise is beneficial for managing type 1 diabetes: it improves insulin sensitivity, reduces cardiovascular risks, improves bones health, etc. However, exercise unbalances glucose homeostasis and may cause hypoglycemia or hyperglycemia, depending on the kind of exercise, its duration, and its intensity. Low-to-moderate aerobic exercise usually lowers glucose; the fear of subsequent hypoglycemia constitutes the main reason people with type 1 diabetes give up an active lifestyle.

Current artificial pancreas systems require patient intervention (they are “hybrid” systems) to counteract meals and exercise. Insulin boluses at mealtime in hybrid artificial pancreas effectively reduce postprandial glucose excursions, but subjects need to timely provide an accurate estimation of the ingested carbohydrate to the system. This estimation is challenging. Then, estimation errors, bolusing delays, or omissions frequently degrade the performance achieved by the system.

Hybrid artificial pancreas systems usually modify glucose reference or basal profile to reduce the impact of exercise, which requires subjects to announce exercise time or intensity even with anticipation.

Some authors have proposed adding carbohydrate suggestions to cope with unannounced exercise events but their proposals still need meal announcements.

Other authors have proposed a meal-detector-based control to remove the meal announcement, but without considering exercise.

Therefore, an improvement is required in current artificial pancreas so as to allow to avoid the need for user intervention in view of meal and exercise events.

### DESCRIPTION OF THE INVENTION

The present invention relates to a method for improving blood glucose control of a hybrid controller. The method of the invention allows to eliminate meal and exercise announcement by substituting patient-initiated meal boluses of said hybrid controller by an automatically generated insulin correction signal without retuning of said hybrid controller, and by incorporating rescue carbohydrates suggestion for hypoglycemia mitigation. The lack of need for retuning of the hybrid controller allows the incorporation of the method of the invention as an extra feature of a system already existing and extensively clinically validated, which can be configured to perform a fully automated mode (activating an add-on module implementing the method of the invention).

Another object of the invention is an add-on module based on an internal model control (IMC) that eliminates meal and exercise announcements from any hybrid controller that includes some restriction of the insulin-on board. The add-on module comprises a calculation unit configured to carry out the method for improving blood glucose control of the hybrid controller.

Another object of the invention is a new artificial pancreas system, which achieves good results in postprandial control. The artificial pancreas system comprises:
a pump (3), for supplying insulin according to a coordinated control action (u_{c});
a continuous glucose monitor (2) for measuring the plasma glucose (G(t)) signal;
a controller for determining the insulin level to be delivered, and
the add-on module described.

Preferably, the module is implemented in an artificial pancreas comprising a controller incorporating a method for limitation of insulin-on-board by means of a safety layer acting on glucose reference so that a tunable upper limit of insulin-on-board is not violated.

In the art, the design of the control algorithm embedded in the hybrid artificial pancreas system, or “main controller”, considers that the user will inform the system about meal intakes and exercise. In the absence of subject announcements, the main controller will likely perform unsatisfactorily.

The module implements an internal model control loop (IMC) that calculates a virtual signal u_{IMC}(t) compensating for the discrepancy between the actual output and an output estimated by a nominal model. Then, a switching logic decomposes this virtual signal in a bolus-like insulin infusion (uᵢₙₛ(t)), correcting the insulin given by the main controller, and rescue carbohydrates suggestions (u_{resc}(t)) to compensate for hyperglycemia and hypoglycemia, respectively.

The switching logic also makes more restrictive the tolerated insulin-on-board after suggesting a rescue carbohydrate intake.

The modification of the tolerated insulin-on-board is the only change the proposed module applies to the internal parameters of the main controller. Most of the hybrid systems constrain the insulin-on-board through gains or thresholds; hence the integration of the module with the main controller is immediate.

The IMC loop requires a glucose-insulin model M. Among the several control-oriented models that could be used in this approach, the Identifiable Virtual Patient (IVP) is preferable because of its structural simplicity and physiological interpretability.

The equations of the model are defined as follows,

$${\dot{I}}_{SC} ( t ) = - \frac{1}{{τ}_{1}} {I}_{SC} ( t ) + \frac{κ}{{τ}_{1} {C}_{1}} {u}_{T} ( t ) \qquad ( 1 a )$$

$${\dot{I}}_{P} ( t ) = - \frac{1}{{τ}_{2}} {I}_{P} ( t ) + \frac{1}{{τ}_{2}} {I}_{SC} ( t ) \qquad ( 1 b )$$

$${\dot{I}}_{EFF} ( t ) = - {p}_{2} {I}_{EFF} ( t ) + {p}_{2} {S}_{I} {I}_{P} ( t ) \qquad ( 1 c )$$

$${\dot{d}}_{1} ( t ) = {A}_{g}^{resc} \cdot {u}_{resc} ( t ) - \frac{{d}_{1} ( t )}{{τ}_{resc}} \qquad ( 1 d )$$

$$\dot{{d}_{2}} ( t ) = \frac{1}{{τ}_{resc}} ( {d}_{1} ( t ) - {d}_{2} ( t ) ) \qquad ( 1 e )$$

$$\dot{G} ( t ) = - GEZI \cdot G ( t ) - {I}_{EFF} ( t ) \cdot G ( t ) + EGP + \frac{{d}_{2} ( t )}{{V}_{g} {τ}_{resc}} \qquad ( 1 f )$$

where I_{SC}(t) and I_{P}(t) are the subcutaneous and plasma insulin concentrations (μU/mL), respectively. State I_{EFF}(t) represents the insulin effect (min⁻¹), and G(t) is the plasma glucose concentration (mg/dL).

The known inputs of the model are the subcutaneous insulin infusion u_{τ}(t) (μU/min) and the rescue carbohydrate suggestion u_{resc}(t) (mg/min).

A two-compartment model, with the glucose masses (mg) d₁(t), d₂(t) as states, models the rescue carbohydrates absorption. The parameters τ₁ and τ₂ (min) stand for the insulin absorption time constants, and p₂ is the kinetic rate for insulin action (min⁻¹). Parameter Cᵢ denotes the insulin clearance (mL/min), Sᵢ represents the insulin sensitivity (mL/μU), EGP is the hepatic glucose production (mg/dL/min), GEZI corresponds to the glucose effectiveness at zero insulin (min⁻¹). Parameter τ_{resc} is the time to the peak absorption of the rescue carbohydrate, and

$${A}_{g}^{resc}$$

is the carbohydrate bioavailability. Finally, κ=60·10⁻⁶ is a factor that converts the units of u_{τ}(t) from μU/min into U/h.

Any other factor affecting glucose (meals aside rescue carbohydrates suggested by the system and exercise among others) will correspond to output disturbances.

Independent of the model M selected, the method for improving blood glucose control of the invention comprises the steps of measuring a plasma glucose (G(t)) signal by means of a continuous glucose monitor (CGM); calculating a glucose level (Ĝ(t)) by using the glucose-insulin model (M), preferably the IVP model, describing the effect of insulin and rescue carbohydrates on glucose; and computing a disturbance term d(t) as:

$$d ( t ) = \hat{G} ( t ) - G ( t )$$

Independent of the model M selected, an IMC filter Q(s) generates a virtual signal u_{IMC}(t) (in insulin units) that mitigates the effect of d(t) on the output. The term d(t) includes everything not modeled by the model M: external disturbances such as the effect of meal intakes and exercise events, and internal disturbances, such as parametric uncertainty in insulin sensitivity or absorption.

Therefore, reducing the effect of d(t) on the output will also attenuate all these disturbances. The IMC filter, Q(s), is selected as in the two-degree-of-freedom IMC:

$$Q ( s ) = \frac{{u}_{IMC} ( s )}{d ( s )} = F ( s ) \cdot {H}^{- 1} ( s ) \qquad ( 2 )$$

where s is the Laplace variable. H(s) is the linearization of the model of Eq. 1 (for u_{resc}(t)=0, i.e., the linearized effect of insulin infusion on glucose when d₁(0)=d₂(0)=0) is preferably given by

$$H ( s ) := \frac{G ( s )}{{u}_{T} ( s )} = \frac{{S}_{I} {G}_{0}^{2}}{{C}_{I} EGP ( \frac{1}{{p}_{2}} s + 1 ) ( {τ}_{1} s + 1 ) ( {τ}_{2} s + 1 ) ( \frac{{G}_{0}}{EGP} s + 1 )} \qquad ( 3 )$$

wherein, if the model M used is the IVP model, G₀ is the steady-state glucose value reached for the patient's basal insulin infusion. The filter F(s) is also preferably defined as:

$$F ( s ) = \frac{k}{{( τ s + 1 )}^{n}} \qquad ( 4 )$$

where k is the gain of the filter. The order of the filter, n, is set so as to Q(s) is strictly proper transfer function when inverting H(s), that is, the degree of the numerator of Q(s) is lower than the degree of the denominator. The time constant τ determines the aggressiveness of the filter. Meal intakes and exercise strongly impact plasma glucose in the short term, but they fade by their dynamics.

In addition, due to absorption and measurement lags, the signal d(t) will acknowledge the onset of actual disturbances (e.g., meals, exercise, etc.) with a delay. If T is set to a high value, the peak of u_{IMC}(t) will occur much after the disturbance peak; hence reducing the disturbance effect by the filter will be negligible and even counterproductive (e.g., in postprandial control, delayed insulin may lead to hypoglycemia). The filter must quickly react against any deviation in d(t) to reduce the effect of disturbances on glucose. For this reason, τ is preferably set to two times the sampling time of the CGM reading rate. More preferably, τ is set to τ=10 min, since the CGM reading rate is usually 5 min.

The time constant τ is set to a low value to counteract the insulin absorption delay by infusing a large amount of insulin in a short time. However, this aggressive tuning amplifies measurement noise, leading to an oscillatory signal u_{IMC}(t). The negative values of u_{IMC}(t) would compensate the positive ones given the lowpass-filter nature of the glucose-insulin system that avoids transferring this measurement noise effect to the output.

However, negative values for insulin are not possible since insulin cannot be removed exogenously. If u_{IMC}(t) were delivered without the negative values would cause an insulin over-delivery, lowering the glucose and even leading to hypoglycemia. Thus, the first goal of the switching logic is to ensure that the proposed loop only applies a control action after a disturbance by removing from u_{IMC}(t) the oscillations caused by measurement noise.

The switching logic also adequates the type of control action to the effect of disturbance on the glucose. Insulin is suitable to compensate for the glucose rise following a meal.

However, aerobic low-to-moderate exercise usually leads to a glucose drop and, eventually, hypoglycemia, which is unlikely to be compensated with only an insulin reduction. To compensate for glucose drop, usually related to exercise and insulin overdoses within the postprandial period, the switching logic reduces the tolerated insulin-on-board and suggests rescue carbohydrates to the subject.

Therefore, the second goal of the switching logic is to convert the virtual signal u_{IMC}(t) into three feedforward actions: insulin infusion, rescue carbohydrate suggestion, and insulin-on-board reduction by:
The insulin uᵢₙₛ(t) is set to 0 if u_{IMC}(t) is lower than a positive threshold thᵢₙₛ to avoid an insulin overdose due to measurement noise amplification in u_{IMC}(t).
Meal ingestion will likely lead to a glucose rise demanding an u_{IMC}(t) that overpasses thᵢₙₛ. To compensate for it, uᵢₙₛ(t) is matched to u_{IMC}(t), to add insulin to the main controller, until it reaches an upper saturation threshold thₛₐₜ, set to avoid overdosing, if u_{IMC}(t) is higher than thᵢₙₛ, or substract insulin from the main controller for negative u_{IMC}(t) higher than th_{resc}.
Against a glucose rise, the IMC filter reacts first with a positive peak (above phase), but then, it will have a negative insulin peak. If u_{IMC}(t) is higher than th_{resc}, uᵢₙₛ(t) will equate u_{IMC}(t) to subtract insulin from the main controller and, hence avoiding overdosing and the likely related hypoglycemia. If u_{IMC}(t) is lower than thᵣₑₛ, reducing the insulin from the main controller may be insufficient. Therefore, the negative-valued insulin is converted into rescue carbohydrates suggestions (u_{resc}(t)), and uᵢₙₛ(t) zeroed to avoid both types of control actions coupling each other.

The switching logic module converts the “negative insulin” into rescue carbohydrate suggestions to mitigate hypoglycemia.

To this end, first, a virtual unquantized carbohydrate signal, uᵢₙₜ(t), is calculated by integrating u_{IMC}(t) in a sliding window of length t_{w}, preferably t_{w}=60 min, as follows:

$${u}_{IMC}^{*} ( t ) = \{ \begin{matrix}{u}_{IMC} ( t ) & if & {u}_{IMC} ( t ) \leq {th}_{resc} \\ 0 & otherwise & \end{matrix} \qquad ( 5 )$$

$${u}_{int} ( t ) = - {k}_{resc} {\int }_{t - {t}_{w}}^{t} {u}_{IMC}^{*} ( τ ) W ( τ ) d τ - {\int }_{t - {t}_{w} - {T}_{s}}^{t - {T}_{s}} {u}_{resc} ( τ ) W ( τ ) d τ \qquad ( 6 )$$

where Tₛ is the sampling time. The first integral in Eq. 6 accumulates the “negative insulin” and transforms it into carbohydrates units (g) through the gain k_{resc}. Noise or unimportant glucose drops may lead to small negative values in u_{IMC}(t), i.e., if −th_{resc}≤u_{IMC}(t)≤0. To avoid suggesting rescue carbohydrates when insulin inhibition may suffice, the first integral in Eq. 6 includes the signal

$${u}_{IMC}^{*} ( t )$$

instead of u_{IMC}(t).

In addition, the forgetting factor W(t) attenuates the earlier values of

$${u}_{IMC}^{*} ( t )$$

in the sliding window. W(t) is defined by a function W(t*) so that:
A) W(t*)=1, for t*=t, being t the current time; and
B) 0≤W(t*)≤1, monotone non-decreasing, for t* in [t−t_{w},t]
   wherein monotone non-decreasing means that W(t₁)≤W(t₂) for all t₁<t₂, and preferably is given by:

$$W ( {t}^{*} ) = {e}^{- 2} \cdot {e}^{( {t}^{*} - t + {t}_{w} ) / 30} \qquad ( 7 )$$

for t+∈[t−t_{w}, t] where t refers to the current time and t−t_{w} the beginning of the sliding window (when the earliest value of

$${u}_{IMC}^{*} ( t )$$

is considered), and t_{w}=60 min. The second integral in Eq. 6 subtracts the rescue carbohydrates suggested within the sliding window to avoid increasing uᵢₙₜ(t).

The virtual carbohydrate signal, uᵢₙₜ(t), must be quantized for user convenience. The quantized rescue signal, u_{resc}(t), follows the next logic:

$${u}_{resc} ( t ) = \{ \begin{matrix}⌊ \frac{{u}_{int} ( t )}{} ⌉ \cdot & if & \begin{matrix}{u}_{int} ( t ) \geq 0.5 and \\ {G}^{*} ( t ) \leq {C}_{2} and Δ {t}_{resc} > {C}_{1}\end{matrix} \\ & if & \begin{matrix}{G}^{*} ( t ) \leq {C}_{3} and \\ CGM ( t ) \leq {C}_{2} and Δ {t}_{resc} > {C}_{1}\end{matrix} \\ 0 & otherwise & \end{matrix} \qquad ( 8 )$$

where └⋅┐ denotes the nearest integer operator, [symbol] is the quantization level, preferably set to 15 g, Δt_{resc}, the elapsed time between two consecutive rescue carbohydrate suggestions (in min). C₁ is a parameter introduced as a delay for avoiding consecutive rescue carbohydrate suggestions, preferably set to 15 min, C₂ and C₃ are parameters related to mild hypoglycemia (also known as level 1 hypoglycemia) and moderate hypoglycemia (also known as level 2 hypoglycemia) thresholds, preferably set to 70 and 54 respectively, and G*(t) is a glucose prediction (in mg/dL), which can be computed with the following linear extrapolation:

$${G}^{*} ( t ) = CGM ( t ) + {C}_{5} \cdot \frac{dCGM ( t )}{dt} \qquad ( 9 )$$

wherein C₅ is a parameter fixing the time of the glucose prediction, preferably set to 30 min.

According to Eq. 8, the controller suggests rescue carbohydrates in two situations:
When the system predicts a moderate hypoglycemia risk and the accumulated rescue carbohydrates are large enough. If uᵢₙₜ(t) halves the minimum rescue dose the algorithm calculates a rescue carbohydrate suggestion by approximating uᵢₙₜ(t) to the nearest multiple of [symbol] , If the predicted glucose is outside hypoglycemia risk, the system will not suggest a rescue carbohydrate even though uᵢₙₜ(t)≥0.5 [symbol] .
When the subject is in moderate hypoglycemia, and the glucose tends to a severe hypoglycemia. Here, the system suggests a rescue regardless of the value of uᵢₙₜ(t)

Preferably [symbol] is set to 15 g since available commercial glucose supplements usually contain this amount, e.g., Dex4 (Can-Am Care, Alpharetta, GA, USA), Glutose15 (Paddock Laboratories, Minneapolis, MN, USA), TruePlus (Trividia Health, Fort Lauderdale, FL, USA), etc. Nevertheless, other quantization than 15 g could have been used.

The algorithm considers a minimum elapsed time of C₃, preferably set to 15 min, between rescue carbohydrates suggestions to avoid frequent recommendations.

After a rescue carbohydrates suggestion, the switching logic:
introduces a more strict limitation of the tolerated insulin-on-board of the main controller;
zeroes uᵢₙₛ(t) during a predefined time T₁ following the last rescue carbohydrate suggestion; and
restores the nominal values of insulin-on-board limitation and uᵢₙₛ(t) when CGM(t) is over a first threshold Th₁ and G*(t) over a second threshold Th₂.

Preferably, the predefined time T₁ is in the range from 30 to 300 min, and the thresholds Th₁ and Th₂ are defined as: CGM(t)≥140 mg/dL and G*(t)≥180 mg/dL

The step of introducing a more strict limitation of the tolerated insulin-on-board of the main controller could be done in different ways such as:
by decreasing an upper limit of insulin-on-board in a given percentage to a percentage between 10% and 90% of its nominal value, preferably 70%,
by adjusting a gain parameter in the controller so that infused insulin is decreased, or
by adjusting the cost of a receding horizon based controller so that infused insulin is decreased.

This module preserved the % time in range achieved by the hybrid artificial pancreas, considering carbohydrate counting errors, without a relevant increase in the time in hypoglycemia. The rescues suggested by the controller counteracted the exercise-induced hypoglycemia, allowing more flexibility than insulin-only controllers.

The method of the invention could also comprise an optimization step for tunning the parameters k, thᵢₙₛ, thₛₐₜ, k_{resc}, th_{resc} by applying a cost defined as:

$${J}_{sim} := {J}_{WAIR} + {J}_{C}$$

The term J_{WAIR} penalizes the weighted areas of the CGM exceeding certain thresholds gᵤᵤ, gᵤ, gₜ and gᵤ as follows:

$$\begin{matrix}{J}_{WAIR} = {a}_{uu} \cdot {\int }_{0}^{Tsim} ( {G}_{uu} ( τ ) - {g}_{uu} ) d τ + {a}_{u} \cdot {\int }_{0}^{Tsim} ( {G}_{u} ( τ ) - {g}_{u} ) d τ + \\ + {a}_{l} \cdot {\int }_{0}^{Tsim} ( {g}_{l} - {G}_{l} ( τ ) ) d τ + {a}_{ll} \cdot {\int }_{0}^{Tsim} ( {g}_{ll} - {G}_{ll} ( τ ) ) d τ + \\ + {a}_{resc} \cdot {\int }_{0}^{Tsim} ( {G}_{resc} ( τ ) - {g}_{resc} ) d τ\end{matrix}$$

Where Tsim is the simulation length and the parameters aᵤᵤ, aᵤ, aₗ, aₗₗ, and, a_{resc} are non-negative scalars defining the weight of each term. The thresholds are positive scalars defined so that gᵤ<gₗ<gᵤ<gᵤᵤ.

Signals Gᵤᵤ(t), Gᵤ(t), Gₗ(t), Gᵤ(t) correspond to the CGM after being saturated to the enclosing thresholds as follows:

$${G}_{uu} ( t ) := \{ \begin{matrix}{g}_{uu} & if & CGM ( t ) \leq {g}_{uu} \\ CGM ( t ) & otherwise & \end{matrix}$$

$${G}_{u} ( t ) := \{ \begin{matrix}{g}_{u} & if & CGM ( t ) \leq {g}_{u} \\ {g}_{uu} & if & CGM ( t ) > {g}_{uu} \\ CGM ( t ) & otherwise & \end{matrix}$$

$${G}_{l} ( t ) := \{ \begin{matrix}{g}_{ll} & if & CGM ( t ) < {g}_{ll} \\ {g}_{l} & if & CGM ( t ) \geq {g}_{l} \\ CGM ( t ) & otherwise & \end{matrix}$$

$${G}_{ll} ( t ) := \{ \begin{matrix}{g}_{ll} & if & CGM ( t ) \geq {g}_{ll} \\ CGM ( t ) & otherwise & \end{matrix}$$

The last addend of J_{WAIR} weights the glucose rebound after rescue carbohydrate suggestion time to better coordinate rescue carbohydrates suggestions and insulin doses.

Signal G_{resc}(t) represents the value of the CGM that overpasses a certain limit of g_{resc} mg/dL in the first C₆ min after rescue carbohydrate suggestions. If a meal occurred before the C₆ min, G_{resc}(t) was calculated until mealtime as defined in:

$${G}_{resc} ( t ) = \{ \begin{matrix}CGM ( t ) & if & \begin{matrix}( CGM ( t ) \geq {g}_{resc} ) and \\ t ∈ [ {t}_{resc} , min ( {t}_{resc} + {C}_{6} , {t}_{meal} ) ]\end{matrix} \\ {g}_{resc} & otherwise & \end{matrix}$$

where t_{resc} and tₘₑₐₗ denote the rescue carbohydrates and meals times, respectively.

The second term of the cost function, i.e., J_{C}, constrains the shape and magnitude of the control actions as defined below:

$$\begin{matrix}{J}_{C} = {b}_{act} \cdot max ( \frac{{n}_{imc \_ act}}{{n}_{meal}} - 1 , 0 ) + {b}_{meal \_ resc} \cdot {\sum }_{i = 1}^{{n}_{meal \_ resc}} {meal \_ resc}_{i} + \\ + {b}_{ex \_ resc} \cdot max ( \frac{{\sum }_{i = 1}^{{n}_{ex \_ resc}} {ex\_resc}_{i}}{{C}_{7} {n}_{ex \_ sessions}} - 1 , 0 )\end{matrix}$$

where:
The first addend penalizes situations wherein the number of IMC activations in insulin mode (n_imc_act) is larger than the number of meals (n_meal) in the optimization scenario. The parameter b_{act} is the weight of the addend and could be any non-negative scalar.
The second addend constrains the total amount of rescues given after meals. The carbohydrate amount given at meal i is defined as meal_resc_i and n__{meal_resc} denotes the number of rescues given after meals. The parameter b__{meal_resc} is the weight of the addend and could be any non-negative scalar.
The third addend penalizes situations wherein the amount of suggested carbohydrate per exercise event exceeds a certain value of C₇ g. The carbohydrate amount suggested after exercise i is denoted as ex_resc_i, and n_ex_resc and n_ex_sessions define the number of exercise-related rescues and exercise events, respectively. The parameter b _{ex_resc} is the weight of the addend and could be any non-negative scalar.

Preferably, the thresholds g_{resc}, gₗₗ, gₗ, gᵤ and gᵤᵤ are in the range from 40 to 400 mg/dL; C₇ in the calculation of G_{resc}(t) is in the range from 5 to 300 min; and the tolerable amount of suggested carbohydrate per exercise event in J_{C}, represented by C₇, is between 5 g and 100 g.

The invention also refers to a computer program adapted for carrying out the steps of the method of the invention defined by using the calculation unit of the add-on module of the invention. The invention also refers to a computer readable storage medium comprising the computer program.

### DESCRIPTION OF THE DRAWINGS

To complement the description being made and in order to aid towards a better understanding of the characteristics of the invention, in accordance with a preferred example of practical embodiment thereof, a set of drawings is attached as an integral part of said description wherein, with illustrative and non-limiting character, the following has been represented:

FIG. 1.—shows an overview of the control method of the invention, wherein blocks with stripped pattern represent the proposed add-on module: an Internal Model Control (IMC) loop (IVP, Identifiable Virtual Patient model, and IMC filter) with a non-linear logic (Switching logic). The add-on module provides three actions: insulin (dashed and double dotted line, uᵢₙₛ(t)), rescue carbohydrates (dotted line, u_{resc}(t)), and maximum insulin on board (IOB) limit command (dashed and dotted line, I̅O̅B̅ (t)).

FIG. 2.—shows the results of a control logic to compensate meals. The switching logic processes the virtual action u_{IMC}(t) in three phases: dead-zone (Phase 1), glucose rise mitigation (Phase 2), and later hypoglycemia prevention (Phase 3). It results in the insulin infusion uᵢₙₛ(t) added to the main controller. Parameters thᵢₙₛ, thₛₐₜ, and th_{resc} are, respectively, the thresholds to inhibit u_{IMC}(t), saturate it, or convert it into rescue carbohydrates suggestions, respectively.

FIG. 3.—shows the results of a control logic to compensate exercise. The switching logic converts the negative insulin of the IMC (u_{IMC}(t), middle panel) into a continuous carbohydrate signal (uᵢₙₜ(t), bottom panel). If the predicted glucose (G*(t), upper panel) is in hypoglycemia and uᵢₙₜ(t)≥7.5 (dashed and dotted line in bottom panel), the algorithm suggests a rescue u_{resc}(t) (bottom panel) by quantizing uᵢₙₜ(t). Hollow squares illustrate that rescue carbohydrates are inhibited if no hypoglycemia risk exists. Insulin uᵢₙₛ(t) is inhibited after the rescue carbohydrate suggestion (stripped pattern area in middle panel)

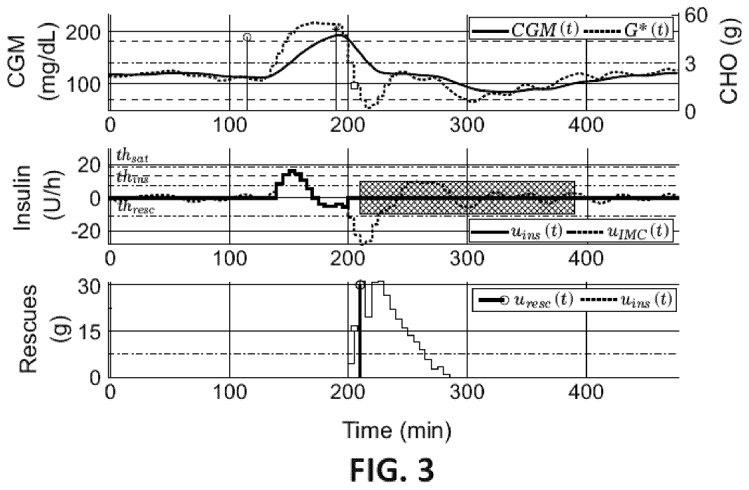

FIG. 4.—shows a mean difference of the percentage time above 180 mg/dL regarding the controller without meal compensation (NoComp). The labels indicate the mean difference between every controller with meal compensation (Hybrid, MD, mIMC) and NoComp obtained using robust random-intercept models. Hybrid represents a controller with meal announcement. MD represents a controller without meal announcement and postprandial hyperglycemia compensation through a meal detection scheme. mIMC represents the method of the invention. Lines represent the Wald 95%-interval confidence. The upper panel refers to the percentage time of CGM above 180 mg/dL within 30 days of simulation, while only the 3 h after each meal are considered in the bottom panel.

FIG. 5.—shows a mean difference of CGM mean, time in range, and daily insulin regarding the controller without meal compensation (NoComp). The labels indicate the mean difference between every controller with meal compensation (Hybrid, MD, mIMC) and NoComp obtained using robust random-intercept models. Hybrid represents a controller with meal announcement. MD represents a controller without meal announcement and postprandial hyperglycemia compensation through a meal detection scheme. mIMC represents the method of the invention. Lines represent the Wald 95%-interval confidence. The upper panel refers to the CGM mean, the middle panel to the percent time in 70-180 mg/dL, and the bottom panel to the daily insulin. All metrics correspond to 30 days of simulation.

FIG. 6.—shows a graph of populational glucose and insulin profiles. It shows 2 of the 30 days of the simulation comparing four meal compensation techniques: absence of meal compensation (NoComp), announced-based compensation (Hybrid), meal-detector-based compensation (MD), and proposed approach (mIMC). The lines represent the median of the 10 virtual adults. The hollow circles in the upper panel represent meal events whose carbohydrate contents are shown on the right axis.

FIG. 7.—shows a mean difference of CGM mean, time in range, and time in hyperglycemia regarding the controller without exercise compensation (NoExComp), that is, the mIMC controller with the rescue carbohydrate suggestion feature deactivated. The labels indicate the estimated mean difference of the controllers MD and mIMC regarding the controller NoExComp obtained using robust random-intercept models. Lines represent the Wald 95%-interval confidence. All metrics correspond to 30 days of simulation.

FIG. 8.—shows a mean difference of time in hypoglycemia regarding the controller without exercise compensation (NoExComp). The labels indicate the estimated mean difference between the controllers MD and mIMC, and the controller NoExComp using robust random-intercept models. Lines represent the Wald 95%-interval confidence. The term “ex” added to the name of the metrics denote that the metric corresponded to the first 3 h after each exercise event, while the remaining metrics considered the 30 days of simulation.

### PREFERRED EMBODIMENT OF THE INVENTION

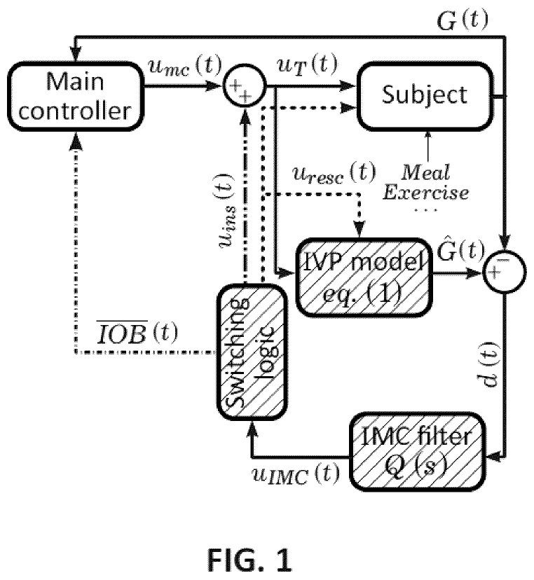

FIG. 1 illustrates a module (see blocks with stripped pattern) that replaces those announcements with only minor modifications in the main controller once plugged into it.

The parameters of the model (Eq. 1) were identified from the 10 virtual adults of the academic version of the UVa/Padova simulator since the controller was evaluated with this cohort. Table 1 includes the identified parameters.

TABLE 1

| | EGP | SI | V g | p₂ |
|---|---|---|---|---|
| Subject | (mg/dL/min) | (mL/μU/min) | (dL) | (1/min) |
| 1 | 1.32 | 5.17 · 10⁻⁴ | 2.35 · 10² | 2.53 · 10⁻³ |
| 2 | 1.20 | 4.24 · 10⁻⁴ | 2.48 · 10² | 4.08 · 10⁻² |
| 3 | 1.05 | 3.35 · 10⁻⁴ | 1.85 · 10² | 2.03 · 10⁻³ |
| 4 | 1.49 | 7.23 · 10⁻⁴ | 2.9 · 10² | 3.39 · 10⁻³ |
| 5 | 0.762 | 2.52 · 10⁻⁴ | 5.81 · 10² | 1.12 · 10⁻² |
| 6 | 0.925 | 2.43 · 10⁻⁴ | 1.83 · 10² | 4.08 · 10⁻² |
| 7 | 0.916 | 2.84 · 10⁻⁴ | 2.54 · 10² | 2.03 · 10⁻³ |
| 8 | 0.925 | 2.39 · 10⁻⁴ | 4.57 · 10² | 6.9 · 10⁻³ |
| 9 | 0.699 | 3.26 · 10⁻⁴ | 2.77 · 10² | 4.08 · 10⁻² |
| 10 | 1.53 | 6.03 · 10⁻⁴ | 2.99 · 10² | 6.01 · 10⁻³ |
| | ${A}_{q}^{resc}$ | CI | GEZI | τ₁ |
| Populational | (unitless) | (mL/min) | (1/min) | (min) |
| | 0.900 | 1.22 · 10³ | 2.35 · 10⁻³ | 74.3 |
| | τ₂ | τ_{rec} | ${A}_{q}^{meal}$ | τₘₑₐₗ |
| | (min) | (min) | (unitless) | (min) |

Control model parameters corresponding to the virtual adults in UVa/Padova simulator. The first column represents the subject identifier in the simulator. Parameters EGP, SI, V g, and p₂ resulted from optimization. Parameters CI, GEZI, T1, and 12 are populational values.
$Parameters {A}_{q}^{resc} and {T}_{resc} were chosen to represent a fast - acting carbohydrate$ $rescue ; they are populational values too . Meal model parameters ( {A}_{q}^{meal} and {T}_{meal} ) .$

The synthetic dataset for identification corresponds to a 2-week basal-bolus therapy of 3 daily meals. Populational values were considered for the absorption dynamics of rescue carbohydrates, and thus parameters in equations Eq. 1d-Eq. 1e were excluded from the identification process.

For the identification of the rest of the model parameters, an additional meal model was considered in order to match clinical data (which include meals).

However, it must be remarked that this meal model was not part of the IMC controller, since meals are unannounced and thus, they are treated as output disturbances. The meal absorption model matches the structure of (1d)-(1e) but with different signals and parameters involved, e.g., larger doses, longer time constants, etc. The identification process used information available in practical settings (CGM reading, insulin infusion, meal dose, and mealtime). Identifiability issues—such as parameter correlation—were handled by selecting the parameters according to the structural identifiability, the global sensitivity, and the collinearity index.

Therefore, the method of the invention relates to a control method having five parameters requiring an individual tuning for the 10 virtual adults in the simulator: the gain of F(s) (kᵢₙₛ), the gain factor converting insulin into carbohydrates (k_{resc}), and the three thresholds of the switching logic (thᵢₙₛ, thₛₐₜ, and th_{resc}). These parameters have been adjusted through the optimization procedure described below.

For each subject, the worst-case within 12 simulations of the same virtual adult, with different instances of parametric variability, is minimized. Each simulation consisted of a 7-day (Tₛᵢₘ=10080 min) scenario with 3 daily meals and 1 daily exercise session.

The cost applied to each simulation is defined as:

$${J}_{sim} := {J}_{WAIR} + {J}_{C} \qquad ( 10 )$$

This cost penalizes the weighted areas in risk (J_{WAIR}) and constrains the magnitude or shape of the control actions (J_{C}). The weighted areas in risk consider the areas of the CGM exceeding the thresholds 54, 70, 180, and 250 as follows:

$$\begin{matrix}{J}_{WAIR} = {a}_{uu} \cdot {\int }_{0}^{Tsim} ( {G}_{uu} ( τ ) - 250 ) d τ + {a}_{u} \cdot {\int }_{0}^{Tsim} ( {G}_{u} ( τ ) - 180 ) d τ + \\ + {a}_{l} \cdot {\int }_{0}^{Tsim} ( 70 - {G}_{l} ( τ ) ) d τ + {a}_{ll} \cdot {\int }_{0}^{Tsim} ( 54 - {G}_{ll} ( τ ) ) d τ + \\ + {a}_{resc} \cdot {\int }_{0}^{Tsim} ( {G}_{resc} ( τ ) - 140 ) d τ\end{matrix} \qquad ( 11 )$$

where the scalars aᵤᵤ=175, aᵤ=1, aₗ=5000, aₗₗ=10000, a_{resc}=50 are the weights. The weights above were tuned so that the areas in hypoglycemia cost more than those in hyperglycemia.

This flexibility in configuring the optimal performance is not possible in other approaches that rely on standard metrics to define the cost. All the integrals were calculated using the trapezoidal rule. Signals Gᵤᵤ(t), Gᵤ(t), Gₗ(t), Gₗₗ(t) in Eq. 11 correspond to the CGM after being saturated to the enclosing thresholds as follows:

$${G}_{uu} ( t ) := \{ \begin{matrix}250 & if & CGM ( t ) \leq 250 \\ CGM ( t ) & otherwise & \end{matrix} \qquad ( 12 )$$

$${G}_{u} ( t ) := \{ \begin{matrix}180 & if & CGM ( t ) \leq 180 \\ 250 & if & CGM ( t ) > 250 \\ CGM ( t ) & otherwise & \end{matrix} \qquad ( 13 )$$

$${G}_{l} ( t ) := \{ \begin{matrix}54 & if & CGM ( t ) < 54 \\ 70 & if & CGM ( t ) \geq 70 \\ CGM ( t ) & otherwise & \end{matrix} \qquad ( 14 )$$

$${G}_{ll} ( t ) := \{ \begin{matrix}54 & if & CGM ( t ) \geq 54 \\ CGM ( t ) & otherwise & \end{matrix} \qquad ( 15 )$$

An insulin overdose might cause a glucose drop that the controller would compensate with rescue carbohydrates suggestions. The last addend of Eq. 11 weights the glucose rebound after rescue carbohydrate suggestion time to better coordinate rescue carbohydrates suggestions and insulin doses.

Signal G_{resc}(t) represents the value of the CGM that overpasses 140 mg/dL in the first 3 h after rescue carbohydrate suggestions. If a meal occurred before the 3 h, G_{resc}(t) was calculated until mealtime as defined in:

$${G}_{resc} ( t ) = \{ \begin{matrix}CGM ( t ) & if & \begin{matrix}( CGM ( t ) \geq 140 ) and \\ t ∈ [ {t}_{resc} , min ( {t}_{resc} + 3 h , {t}_{meal} ) ]\end{matrix} \\ 140 & otherwise & \end{matrix} \qquad ( 16 )$$

where t_{resc} and tₘₑₐₗ denote the rescue carbohydrates and meals times, respectively.

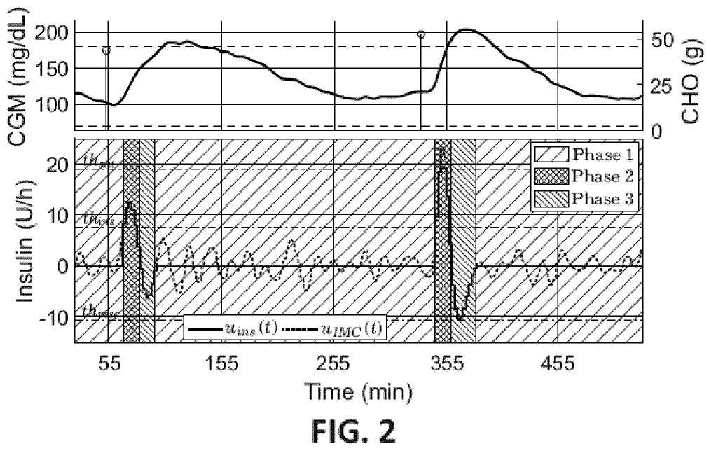

Mealtimes were available to define the cost function but were unknown to the controller. The cost J_{C} penalizes the number of times the IMC activates the insulin mode (Phase 2 of FIG. 2) for uᵢₙₛ(t) to behave like a bolus: being active a short time with large insulin doses rather than being continuously activated with reduced doses. The cost J_{C} also constrains the size of rescue carbohydrates. To reduce the risk of compensating insulin overdosing with rescue carbohydrates, the carbohydrate suggestions followed by meals (meal rescue carbohydrates) were weighted more than those followed by exercise sessions (exercise rescue carbohydrates). For the exercise-related rescue carbohydrates, the average rescue size per exercise event was limited to 45 g. The expression of J_{C} is the following:

$$\begin{matrix}{J}_{C} = {b}_{act} \cdot max ( \frac{{n}_{imc \_ act}}{{n}_{meal}} - 1 , 0 ) + {b}_{meal \_ resc} \cdot {\sum }_{i = 1}^{{n}_{meal \_ resc}} {meal \_ resc}_{i} + \\ + {b}_{ex \_ resc} \cdot max ( \frac{{\sum }_{i = 1}^{{n}_{ex \_ resc}} {ex\_resc}_{i}}{45 {n}_{ex \_ sessions}} - 1 , 0 )\end{matrix} \qquad ( 17 )$$

where b_{act}=1400, b_{meal_resc}=15000, and b_{ex_resc}=4500 are weights. Terms n_{imc_act}, n_{meal_resc}, n_{ex_resc}, n_{ex_session} denote the number of times the IMC enters Phase 2, the number of meal-related rescue carbohydrates, the number of exercise-related rescue carbohydrates, and the number of exercise sessions, respectively. meal_rescᵢ represents the meal rescue sizes (from i=1 to i=n_{meal_resc}) and meal_exᵢ the exercise rescue sizes (from i=1 to i=n_{ex_resc}).

The min-max problem was solved with the Covariance Matrix-Adaptation Evolution Strategy (CMA-ES) algorithm, a black-box search optimizer suitable for non-linear or non-convex problems. Table 2 includes the starting values and the bounds of the parameters.

TABLE 2

| | Initial value | Lower limit | Upper limit |
|---|---|---|---|
| kᵢₙₛ (−) | 0.5 | 0.01 | 1 |
| thᵢₙₛ (U/h) | 5 | 1 | 30 |
| thₛₐₜ (U/h) | 10 | 1 | 30 |
| th_{resc} | 1 | 0.05 | 5 |
| (U/h) | | | |
| k_{resc} | 0.1 | 0.0005 | 0.5 |
| (g/U/h) | | | |

The above method of tuning the controllers is feasible for in-silico studies. To provide a starting tuning for a clinical trial, the optimal parameters were related by means of a regression to standard parameters of the open-loop therapy: the weight (BW, in kg), the total daily insulin (TDI, in U), the basal insulin (u_{b}, in U/h), the carbohydrate-to-insulin ratio (CR, in g/U), and the correction factor (CF, in mg/dL/U). For each optimal parameter, a relation to open-loop parameters was found as follows:
1. The 80 linear models that fit the corresponding optimal parameter with the lowest root sum of squares were selected. Models had up to 8 coefficients, including pairwise interactions of the open-loop parameters. The selection was performed with the function regsubset of the R software.
2. To mitigate the risk of overfitting, the selected models were fitted using leave-one-out cross-validation.
3. The final model was the model with the lowest number of coefficients that had an accurate root-mean-squared error of the cross-validation and satisfied the diagnosis assumptions (normality and homoscedasticity of the residuals).

The proposed add-on module was implemented in the UVA/Padova simulator for validation purposes. The simulator emulates the 5-min sampling time of the CGM; hence the add-on module must be implemented in discrete time.

For the implementation, the model in Eq. 1 was discretized with Euler approximation using a sampling period of 5 min (Tₛ=5 min). The filter Q(s) in Eq. 2 and the integral in Eq. 6 were discretized using a Tustin approximation, also with Tₛ=5 min.

The validation targets three purposes:
1) to determine whether the regression-based tuning maintains the performance of the optimal tuning,
2) to assess the controller against meals, and
3) to assess the controller against meals and exercise.

The fit of the regression model to the corresponding optimal parameter was assessed with the coefficient of determination R² and the root-mean-squared error of the cross-validation (RMSE_{loocv}).

To study if the regression-based tuning degraded the performance of the optimal tuning, glucose percentage time-related metrics were compared(the % time in range, the % time in hyperglycemia, and the % time in hypoglycemia).

To this end, both tunings were simulated for 10 virtual adults using the UVa/Padova simulator.

The simulation consisted of a 30-day scenario including 3 daily meals, with random sizes and timing, and 1 daily exercise session. Defining the daily meals by:
For breakfast: a median of 49.5 g, with an interquartile range of 33.0 g, 55.0 g at a median of 6.92 h and at an interquartile range of 6.75 h, 7.08 h.
For lunch: a median of 81.0 g, with an interquartile range of 72.0 g, 93.0 g at a median of 13.75 h and at an interquartile range of 13.58 h, 14.17 h.
For dinner: a median of 64 g, with an interquartile range of 54 g, 79 g at a median of 20.9 h and at an interquartile range of 20.8 h, 21.1 h.

Exercise effect on glucose was simulated through a variation of insulin sensitivity. This exercise model corresponds to an aerobic exercise of 60 min at 50% of VO₂ₘₐₓ, approximately. The exercise time was set up to 240 min after one meal of the day—12 exercise events after breakfast, 11 after lunch, and 7 after dinner—following a uniform distribution. Exercise events beyond midnight were avoided.

The simulation also included multiple sources of variability: CGM noise, one-day period sinusoidal-type insulin sensitivity variation with random amplitude and phase, variation of subcutaneous insulin absorption rate at each meal following a uniform distribution of ±30%, and variability of the meal absorption parameters.

The goal of the validation performance against meals is to quantize the improvement regarding the main controller without any meal compensation (henceforth denoted as NoComp) of three controllers with meal compensation: 1) the main controller with the IMC loop, tuned with the regression model (denoted as mIMC), 2) the main controller with the meal-announcement free compensation feature, based on a super-twisting meal detector (referred as MD), and 3) the main controller with meal announcements but considering errors in the estimation of the carbohydrates (denoted as Hybrid).

The simulation features—duration, number of subjects, variability, meal size, and timing-were identical to the ones described before, but without considering exercise.

To assess the controllers, apart from the standard metrics, percentage-time-related metrics within the postprandial period (from the mealtime until 3 h after each meal) were calculated. Since the mIMC might compensate insulin over-delivery with rescue carbohydrates suggestions, the percentage of meals requiring at least one rescue and the mean size of the rescue carbohydrates suggested for those meals were also reported.

A validation performance against exercise is also carried out, being assessed the likely benefits of the rescue carbohydrate suggestion feature of the mIMC to counteract exercise-induced hypoglycemia.

To this end, the proposed controller mIMC was compared to two insulin-only controllers: 1) the mIMC controller with the rescue carbohydrate suggestion feature deactivated (denoted as NoEx-Comp), and 2) the meal-detector based controller, i.e., MD.

The simulation scenario was identical to the one described before, with exercise events. Apart from the standard metrics, the following exercise-related metrics were computed: the % time in hypoglycemia within the exercise period (from the exercise time to 3 h after it), the % time above 140 mg/dL up to 3 h after each rescue, the percentage of exercise events needing at least one rescue carbohydrate, and the mean rescue size suggested for those events.

In the simulations, subjects ingested the suggested carbohydrates in the precise time and size.

Results were analyzed with a regression-based inference approach and Wald 95% confidence intervals. Since all the simulations in the study shared the virtual cohort, the independence condition assumed by linear models fails.

Mixed-effect models can handle the dependency of the virtual subjects in the sample. For each of the analyzed metrics, the following random-intercept mixed-effect model, individualized for each subject, was fitted:

$${y}_{sub} = {β}_{0} + {S}_{sub} + {\sum }_{i = 1}^{{n}_{C} - 1} {β}_{i} {x}_{i} + {e}_{sub} \qquad ( 18 )$$

where y_{sub} is the corresponding metric value for a given subject, sub, and n_{C} is the number of controllers to be compared. S_{sub} is the random intercept, and e_{sub} are the residuals, following both a zero-mean normal distribution. Fixed coefficient β₀ is the intercept, and coefficients βᵢ can be interpreted as the mean difference regarding the intercept since xᵢ is a dichotomous dummy variable related to the controller to be compared with the intercept. These coefficients inform about the effect size of the differences between structures, a more valuable information than the significance analysis of P-values, especially for in silico analysis where P-values are controversial.

The mixed-effect model was fitted with a robust method of the R software to handle the outliers appearing in the data.

Table 3 includes the optimal parameters of the controller described before. The related regression equations (Table 4) accurately fit the optimal parameters.

TABLE 3
The first column represents the virtual adult identifier in the
UVa/Padova Simulator. The second, third, and fourth columns
include the parameters used for meal compensation, while the
remaining columns correspond to the exercise compensation.

| | kᵢₙₛ | thₘᵢₙ | thₘᵢₙ | k_{resc} | th_{resc} |
|---|---|---|---|---|---|
| Subject | (−) | (U/h) | (U/h) | (g/U/h) | (U/h) |
| 1 | 0.04 | 4.43 | 9.38 | 0.09 | 0.48 |
| 2 | 0.17 | 1.46 | 14.34 | 0.31 | 0.15 |
| 3 | 0.03 | 1.08 | 13.20 | 0.06 | 0.53 |
| 4 | 0.08 | 5.50 | 12.19 | 0.12 | 0.07 |
| 5 | 0.11 | 3.72 | 25.95 | 0.09 | 0.07 |
| 6 | 0.25 | 7.57 | 19.62 | 0.12 | 2.08 |
| 7 | 0.02 | 5.42 | 10.25 | 0.09 | 0.12 |
| 8 | 0.14 | 6.83 | 15.22 | 0.05 | 2.45 |
| 9 | 0.34 | 2.34 | 8.08 | 0.16 | 0.72 |
| 10 | 0.14 | 9.69 | 15.69 | 0.09 | 0.35 |

TABLE 4
Regression equations of the controller's parameters and related goodness
of t metrics, which are the adjusted coefficient of determination (adjR2)
for multivariable regression models and root-mean-squared error of the
leave-one out cross-validation (RMSEloocv). The five models have a low
RMSEloocv and acceptable coefficients of determination.

| Subject | adjR² | RMSE_{loocv} |
|---|---|---|
| \hat{k} = 14.2 + 6.17 · 10⁻² · TDI − 2.59 · u_{b} − −1.61 · | 0.972 | 0.0471 |
| 10⁻³ · BW · CF + 3.93 · 10⁻³ · BW · CR − −4.67 · | | |
| 10⁻³ · CF · TDI − 6.57 · 10⁻³ · CR · TDI | | |
| [symbol] = −51.6 + 28.9 · CR + 0.872 · BW · u_{b} − −2.53 · | 0.752 | 1.95 |
| 10⁻² · BW · CF − 12.9 · CR · u_{b} − −0.226 · CF · CR | | |
| [symbol] = 4.01 · 10² − 1.05 · 10² · u_{b} − 20.8 · CR − −8.82 · | 0.890 | 3.16 |
| 10⁻² · BW · CF + 0.209 · BW · CR + +0.150 · CF · CR | | |
| \hat{k}_{resc} = −3.02 − 7.6 · 10⁻² · CR + 3.11 · 10⁻⁴ · BW · TDI − −1.74 · | 0.776 | 0.0676 |
| 10⁻² · BW · u_{b} + 1.76 · 10⁻⁴ · BW · CF + +8.18 · 10⁻² · CF · u_{b} | | |
| [symbol] = −12.3 − 0.133 · BW − 0.295 · TDI + +0.103 · | 0.955 | 0.209 |
| BW · u_{b} + 1.48 · 10⁻² · CF · TDI − −2.55 · 10⁻³ · CF · CR | | |

Furthermore, the mean difference in the metrics % time in 70-180 mg/dL (0.303%, CI:[−1.30, 1.91]), % time above 180 mg/dL (−0.470%, CI:[−2.29, 1.35]), and % time below 70 mg/dL (0.0576%, CI:[−0.199, 0.315]) are negligible, which indicate that the regression-based tuning preserves the performance achieved by the optimal tuning.

The controllers featuring meal compensation (Hybrid, MD, or mIMC) outperform NoComp, as shown in Table 5.

TABLE 5
Performance metrics of meal compensation. Four meal compensation techniques,
which share the same main controllers, were compared: absence of meal compensation
(NoComp), announced-based compensation (Hybrid), meal-detector-based compensation
(MD), and proposed approach (mIMC). Metrics are expressed in median [25th
percentile, 75th percentile] of the 10 virtual adults. Overall metrics
aggregate the entire simulation period (30 days), while Postprandial control
metrics refer to a specific period of the postprandial: percent of time-
related metrics aggregate the 3-h period after the meal, and rescue-related
metrics aggregate the meal-to-meal period.

| | NoComp | Hybrid | MD | mIMC |
|---|---|---|---|---|
| Overall | | | | |
| Mean CGM | 161.6 [158.9, | 140.6 [139.0, | 141.8 [139.0, | 140.0 [132.9, |
| (mg/dL) | 189.5] | 154.2] | 145.6] | 144.8] |
| CV (%) | 30.5 [28.7, | 25.2 [23.4, | 25.8 [24.4, | 25.2 [24.4, |
| | 33.7] | 26.5] | 29.2] | 28.6] |
| % of time CGM | | | | |
| >250 mg/dL | 8.2 [4.8, | 1.2 [0.3, | 1.9 [0.7, | 1.4 [0.3, |
| (%) | 19.5] | 2.3] | 4.0] | 2.3] |
| >180 mg/dL | 31.8 [29.9, | 16.0 [13.6, | 17.3 [16.2, | 14.7 [11.8, |
| (%) | 45.2] | 24.1] | 20.4] | 22.1] |
| 70-180 mg/dL | 68.2 [54.8, | 84.0 [75.9, | 81.9 [79.3, | 85.1 [77.9, |
| (%) | 70.1] | 86.4] | 83.8] | 88.1] |
| <70 mg/dL | 0.0 [0.0, | 0.0 [0.0, | 0.0 [0.0, | 0.0 [0.0, |
| (%) | 0.0] | 0.0] | 0.1] | 0.1] |
| <54 mg/dL | 0.0 [0.0, | 0.0 [0.0, | 0.0 [0.0, | 0.0 [0.0, |
| (%) | 0.0] | 0.0] | 0.0] | 0.0] |
| Daily insulin (U) | 34.7 [29.8, | 38.6 [34.0, | 38.3 [32.9, | 38.5 [34.1, |
| | 37.4] | 41.3] | 39.9] | 41.4] |
| Postprandial | | | | |
| control | | | | |
| % of time CGM | | | | |
| >250 mg/dL | 17.5 [9.4, | 2.6 [0.9, | 5.1 [1.7, | 3.5 [0.7, |
| (%) | 28.3] | 4.2] | 9.4] | 5.9] |
| >180 mg/dL | 57.2 [49.1, | 34.0 [25.7, | 41.4 [37.6, | 34.9 [29.4, |
| (%) | 62.3] | 35.5] | 47.5] | 45.5] |
| <70 mg/dL | 0.0 [0.0, | 0.0 [0.0, | 0.0 [0.0, | 0.0 [0.0, |
| (%) | 0.0] | 0.0] | 0.0] | 0.0] |
| <54 mg/dL | 0.0 [0.0, | 0.0 [0.0, | 0.0 [0.0, | 0.0 [0.0, |
| (%) | 0.0] | 0.0] | 0.0] | 0.0] |
| Meals needing | — | — | — | 0.0 [0.0, |
| rescues(%) | | | | 1.1] |
| Mean rescues | — | — | — | 15.0 [15.0, |
| (g) | | | | 15.0] |

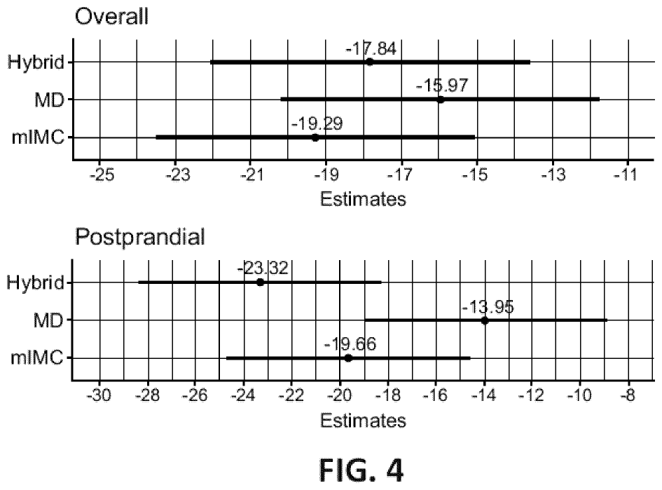

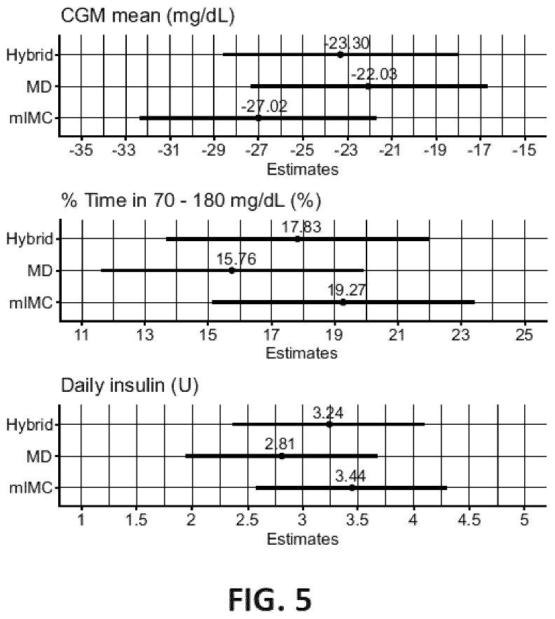

The improvement is statistically significant since all the fixed-effect coefficients of FIG. 4 and FIG. 5—interpreted as the mean difference of each controller regarding NoComp—are far from zero.

Since the confidence intervals in FIG. 4 and FIG. 5 overlap among controllers, all the controllers with meal compensation improve to a similar degree the performance of NoComp, with the advantage that the MD and mIMC controllers free subjects from meal announcements.

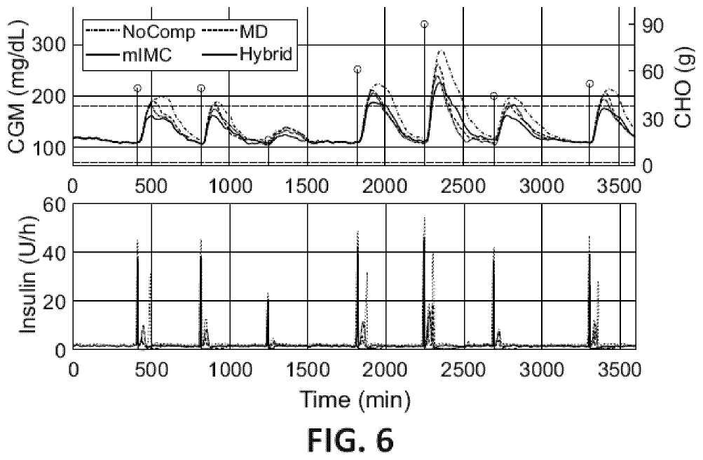

FIG. 6 shows the glucose and insulin traces of 2 of the 30 days of the simulation. Some behavioral differences exist between the controllers. The meal announcement in the hybrid controller improves the early phase of the postprandial with a lower time in hyperglycemia, as seen in the bottom panel of FIG. 4, and a lower postprandial peak, as seen in FIG. 6.

The mIMC tends to be more aggressive, allowing a more rapid recovery than the hybrid controller after large meal, as shown in fifth meal in FIG. 6. The price to pay to achieve a similar time in range than the hybrid controller, but without announcement, is a steeper glucose drop after the postprandial (FIG. 6).

In no subject, the glucose drops below 54 mg/dL and the % time below 70 mg/dL never overpasses the 0.5%, which is far from the accepted 4% threshold indicated in the literature.

Although the rescue suggestion feature plays a role in avoiding hypoglycemia events, its use was sparse: for most of the subjects, the controller did not suggest any rescue; for two of them, the controller only recommended 15 g; and only for the two remaining ones, the controller recommended more than one rescue, 10 and 12 rescues, always of 15 g.

Finally, the MD achieves a slightly longer time in hyperglycemia than the mIMC, as shown in Table 5. Since the MD does not suggest rescues, it cannot mitigate the glucose drop. As a result, unlike the mIMC, one virtual subject had severe hypoglycemia.

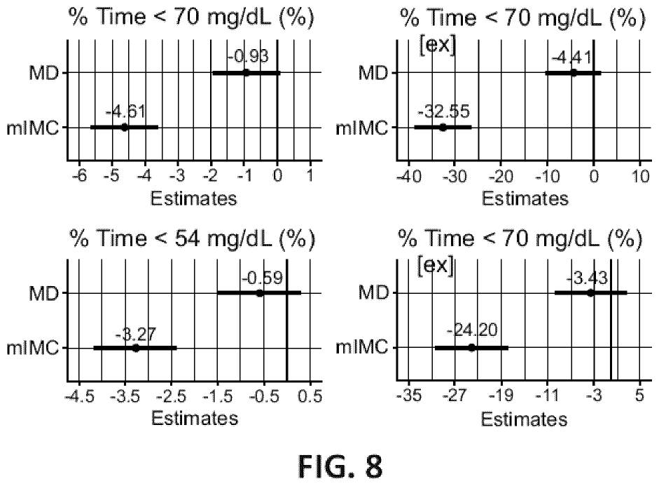

The results on the performance of exercise control are also shown. The carbohydrate suggestion feature in the mIMC significantly reduces the time in moderate and severe hypoglycemia, as seen in FIG. 8, compared to when the rescue module is unable (NoExComp).

The meal-detector-based controller (MD) performed like NoExComp as concluded from the confidence intervals, they are small and include the 0, as can be seen in Table 6 and FIG. 8). Therefore, the flexibility of insulin-only controllers against exercise-induced hypoglycemia is limited.

The mIMC suggested a median of 27.2 g per exercise session to handle the exercise-induced glucose drop, as shown in Table 6. This value is coherent with other results of unannounced exercise events in the literature.

TABLE 6
Performance against exercise. It includes the results of three controllers: the meal-detector-
based controller (MD), the proposed controller (mIMC), and the proposed controller disabling
the rescue suggestion module (NoExComp). Metrics are expressed in median [25th percentile,
75th percentile] of the 10 virtual adults. Overall metrics aggregate the entire simulation
period (30 days), while Exercise control metrics refer to a specific period after the
exercise: percent of time-related metrics aggregate the 3-h period after the exercise,
and rescue-related metrics aggregate the exercise-to-exercise period.

| | NoExComp | | MD | | mIMC | |
|---|---|---|---|---|---|---|
| Overall | | | | | | |
| Mean CGM (mg/dL) | 138.2 | [132.1, 141.7] | 136.9 | [135.6, 138.5] | 141.9 | [136.5, 146.2 |
| CV (%) | 33.3 | [31.4, 34.4] | 34.1 | [33.1, 35.1] | 29.6 | [27.4, 32.0] |
| % of time CGM | | | | | | |
| >250 mg/dL (%) | 1.6 | [0.8, 2.9] | 2.2 | [1.5, 3.6] | 1.6 | [1.0, 2.9] |
| >180 mg/dL (%) | 19.2 | [13.5, 21.1] | 19.2 | [18.1, 20.6] | 19.8 | [13.6, 20.8] |
| 70-180 mg/dL (%) | 75.5 | [72.7, 81.5] | 76.5 | [71.6, 77.0] | 79.6 | [77.5, 85.5] |
| <70 mg/dL (%) | 5.3 | [4.5, 6.6] | 4.6 | [4.0, 5.1] | 0.9 | [0.4, 1.1] |
| <54 mg/dL (%) | 3.7 | [2.2, 4.1] | 2.8 | [2.0, 3.4] | 0.0 | [0.0, 0.0] |
| Daily insulin (U) | 37.9 | [33.0, 40.7] | 37.6 | [31.8, 39.6] | 37.7 | [33.2, 40.6] |
| Daily CHO (g) | 0.0 | [0.0, 0.0] | 0.0 | [0.0, 0.0] | 27.5 | [23.9, 31.0] |
| Exercise control | | | | | | |
| % of time CGM | | | | | | |
| >140 mg/dL (%) | — | | — | | 9.6 | [5.6, 12.8] |
| (resc) | | | | | | |
| <70 mg/dL (%) | 38.3 | [34.6, 44.1] | 34.7 | [30.7, 39.2] | 6.5 | [3.0, 8.7] |
| <54 mg/dL (%) | 27.0 | [17.0, 30.4] | 21.6 | [16.1, 26.4] | 0.1 | [0.0, 0.3] |

| | | | | |
|---|---|---|---|---|
| Events with rescues | — | — | 96.7 | [96.7, 96.7] |
| (%) | | | | |
| Mean rescues (g) | — | — | 27.2 | [23.7, 31.0] |

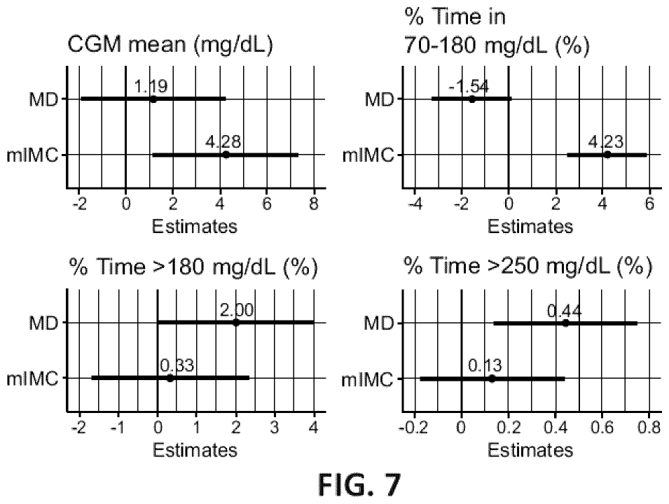

Even with the additional carbohydrate intake, the controller achieved a time in hyperglycemia similar to the NoExComp, as shown in FIG. 7. The mIMC increases the CGM mean to 4.28 mg/dL (CI:2.90-5.91) mg/dL on average; although statistically significant, this increase, concerning NoExComp, has a minor relevance from the clinical point of view. The rise in the % time above 140 mg/dL after the rescue carbohydrate suggestion is also permissible (Table 6).

1. Method for improving blood glucose control of a hybrid controller, eliminating meal and exercise announcement by substituting patient-initiated meal boluses of the hybrid controller by an automatic insulin correction signal without retuning of said hybrid controller, and incorporating rescue carbohydrates suggestion, the method comprising the steps of:
measuring a plasma glucose (G(t)) signal by means of a continuous glucose monitor (CGM);
calculating a glucose level (Ĝ(t)) by using a glucose-insulin model (M) describing the effect of insulin and rescue carbohydrates on glucose;
computing a disturbance term d(t) as:
$$d ( t ) = \hat{G} ( t ) - G ( t )$$
generating a virtual signal u_{IMC}(t), for mitigating the effect of d(t) on the output, by means of an IMC filter Q(s), defined as:
$$Q ( s ) = \frac{{u}_{IMC} ( s )}{d ( s )} = F ( s ) \cdot {H}^{- 1} ( s )$$
wherein s is the Laplace variable, H(s) is given by the linearization of the model M with respect to insulin input, and filter F(s) is defined so that the degree of the numerator of Q(s) is lower than the degree of the denominator of Q(s);
converting the virtual signal u_{IMC}(t) into three feedforward actions: insulin infusion, rescue carbohydrate suggestion, and insulin-on-board reduction by:
setting insulin uᵢₙₛ(t) to 0 if u_{IMC}(t) is lower than a positive threshold thᵢₙₛ;
matching uᵢₙₛ(t) to u_{IMC}(t), to add insulin to the main controller, limited by an upper saturation threshold thₛₐₜ, if u_{IMC}(t) is higher than thᵢₙₛ, or subtract insulin from the main controller, when u_{IMC}(t) is below the negative threshold th_{resc}:
converting the negative-valued insulin u_{IMC}(t) into rescue carbohydrates suggestions (u_{resc}), then zeroing uᵢₙₛ(t) to avoid both types of control actions coupling each other if u_{IMC}(t) is lower than th_{resc}, comprising conversion of negative insulin into rescue carbohydrate suggestions the steps of:
generating a virtual unquantized carbohydrate signal, uᵢₙₜ(t), integrating u_{IMC}(t) in a sliding window of length t_{w}, as follows:
$${u}_{int} ( t ) = - {k}_{resc} {\int }_{t - {t}_{w}}^{t} {u}_{IMC}^{*} ( τ ) W ( τ ) d τ - {\int }_{t - {t}_{w} - {T}_{s}}^{t - {T}_{s}} {u}_{resc} ( τ ) W ( τ ) d τ$$
using a variable
$${u}_{IMC}^{*}$$
instead of u_{IMC} to avoid suggesting rescue carbohydrates when insulin inhibition may suffice, calculated as:
$${u}_{IMC}^{*} ( t ) = \{ \begin{matrix}{u}_{IMC} ( t ) & if & {u}_{IMC} ( t ) \leq {th}_{resc} \\ 0 & otherwise & \end{matrix}$$
and where Tₛ is the sampling time, k_{resc} is a gain for converting “negative insulin” into rescue carbohydrate suggestions and W(t*) is a forgetting factor that attenuates the earlier values of
$${u}_{IMC}^{*} ( t )$$
by using a monotone non-decreasing function W(t*), which has values for t*∈[t−t_{w}, t], where t refers to the current time and t−t_{w} the beginning of the sliding window, and so that W(t)=1
calculating u_{resc}(t) by:
$${u}_{resc} ( t ) = \{ \begin{matrix}⌊ \frac{{u}_{i n t} ( t )}{} ⌉ \cdot & \begin{matrix}if {u}_{i n t} ( t ) \geq 0.5 and \\ {G}^{*} ( t ) \leq {C}_{2} and Δ {t}_{resc} > {C}_{1}\end{matrix} \\ & \begin{matrix}if {G}^{*} ( t ) \leq {C}_{3} and \\ CGM ( t ) \leq {C}_{2} and Δ {t}_{resc} > {C}_{1}\end{matrix} \\ 0 & otherwise\end{matrix}$$
wherein └⋅┐ denotes the nearest integer operator, [symbol] is the quantization level, Δt_{resc} is the elapsed time between two consecutive rescue carbohydrate suggestions, C₁ is a parameter introduced as a delay for avoiding consecutive rescue carbohydrate suggestions, C₂ and C₃ are parameters related to mild and moderate hypoglycemia thresholds, and G*(t) is a glucose prediction.

2. Method according to claim 1, wherein the glucose-insulin model is the Identifiable Virtual Patient (IVP) defined by:
$${\dot{I}}_{S C} ( t ) = - \frac{1}{{τ}_{1}} {I}_{S C} ( t ) + \frac{K}{{τ}_{1} {C}_{I}} {u}_{T} ( t )$$
$${\dot{I}}_{P} ( t ) = - \frac{1}{{τ}_{2}} {I}_{P} ( t ) + \frac{1}{{τ}_{2}} {I}_{s c} ( t )$$
$${\dot{I}}_{E F F} ( τ ) = - {p}_{2} {I}_{E F F} ( t ) + {p}_{2} {S}_{I} {I}_{P} ( t )$$
$${\dot{d}}_{1} ( t ) = {A}_{g}^{resc} \cdot {u}_{resc} ( t ) - \frac{{d}_{1} ( t )}{{τ}_{resc}}$$
$${\dot{d}}_{2} ( t ) = \frac{1}{{τ}_{resc}} ( {d}_{1} ( t ) - {d}_{2} ( t ) )$$
$$\dot{G} ( t ) = GEZI \cdot G ( t ) - {I}_{EFF} ( t ) \cdot G ( t ) + EGP + \frac{{d}_{2} ( t )}{{V}_{g} {τ}_{resc}}$$
where I_{SC}(t) and I_{P}(t) are the subcutaneous and plasma insulin concentrations (μU/mL), respectively, I_{EFF}(t) represents the insulin effect, and G(t) is the plasma glucose concentration (mg/dL), the subcutaneous insulin infusion u_{τ}(t) (μU/min) and the rescue carbohydrate suggestion u_{resc}(t) (mg/min) are defined, and a two-compartment model, with the glucose masses (mg) d1(t), d2(t) as states, models the rescue carbohydrates absorption, also, the parameters τ1 and τ2 (min) stand for the insulin absorption time constants, and p2 is the kinetic rate for insulin action, parameter Cₗ denotes the insulin clearance (mL/min), Sₗ represents the insulin sensitivity (mL/μU), EGP is the hepatic glucose production (mg/dL/min), GEZI corresponds to the glucose effectiveness at zero insulin (min⁻¹), parameter τ_{resc} is the time to the peak absorption of the rescue carbohydrate,
$${A}_{g}^{resc}$$
is the carbohydrate bioavailability, and κ=60·10⁻⁶ is a conversion factor.

3. Method according to claim 2, where the linearized model H(s) is computed as:
$$H ( s ) := \frac{G ( s )}{{u}_{T} ( s )} = \frac{{S}_{I} {G}_{0}^{2}}{{C}_{I} EGP ( \frac{1}{{p}_{2}} s + 1 ) ( {τ}_{1} s + 1 ) ( {τ}_{2} s + 1 ) ( \frac{{G}_{0}}{E G P} s + 1 )} .$$

4. Method according to claim 1, wherein the parameter C₁ for avoiding consecutive rescue carbohydrate suggestions is 15 min.

5. Method according to claim 1, wherein the monotone non-decreasing function W(t*), is represented by an exponential expression of the form:
$$W ( {t}^{*} ) = {C}_{4} \cdot {e}^{- \frac{( {t}^{*} - t + {t}_{w} ) ln ( {C}_{4} )}{{t}_{w}}}$$
for t*∈[t−t_{w}, t], where t refers to the current time and t−t_{w} the beginning of the sliding window, with C₄ being a constant that states the level of
$${u}_{IMC}^{*}$$
(t) and verifies 0<C₄≤1; and ln (C₄) denotes the natural logarithm of C₄.

6. Method according to claim 1, wherein the sliding window, is given by the forgetting factor W(t*) defined as:
$$W ( {t}^{*} ) = {e}^{- 2} \cdot {e}^{( {t}^{*} - t + 60 ) / 30} .$$

7. Method according to claim 1, wherein the parameters C₂ and C₃ related to hyperglycemia and hypoglycemia thresholds are 70 and 54, respectively.

8. Method according to claim 1, wherein the glucose prediction G*(t) is computed with the following linear extrapolation:
$${G}^{*} ( t ) = CGM ( t ) + {C}_{5} \cdot \frac{dCGM ( t )}{d t}$$
wherein C₅ is a parameter fixing the time of the glucose prediction to 30 min.

9. Method according to claim 1, wherein the filter F(s) is defined as:
$$F ( s ) = \frac{k}{{( τ s + 1 )}^{n}}$$
where k is the gain of the filter, n is high enough so that the degree of the numerator of Q(s) is lower than the degree of the denominator of Q(s), and t is a time constant which determines the aggressiveness of the filter.

10. Method according to claim 1, wherein, after a rescue carbohydrates suggestion, the switching logic:
introduces a more strict limitation of the tolerated insulin-on-board of the main controller by decreasing an upper limit of insulin-on-board in a given percentage to a percentage between 10% and 90% of its nominal value;
zeroes uᵢₙₛ(t) during a predefined time T₁ following the last rescue carbohydrate suggestion; and
restores the nominal values of insulin-on-board limitation and uᵢₙₛ(t) when CGM(t) is over a first threshold Th₁ and G*(t) over a second threshold Th₂.

11. Method according to claim 10, wherein the percentage of reduction is set to 70% of its nominal value.

12. Method according to claim 1, wherein, after a rescue carbohydrates suggestion, the switching logic:
introduces a more strict limitation of the tolerated insulin-on-board of the main controller by adjusting a gain parameter in the controller so that infused insulin is decreased;
zeroes uᵢₙₛ(t) during a predefined time T₁ following the last rescue carbohydrate suggestion; and
restores the nominal values of insulin-on-board limitation and uᵢₙₛ(t) when CGM(t) is over a first threshold Th₁ and G*(t) over a second threshold Th₂.

13. Method according to claim 1, where after a rescue carbohydrates suggestion, the switching logic:
introduces a more strict limitation of the tolerated insulin-on-board of the main controller by adjusting the cost of a receding horizon based controller so that infused insulin is decreased;
zeroes uᵢₙₛ(t) during a predefined time T₁ following the last rescue carbohydrate suggestion; and
restores the nominal values of insulin-on-board limitation and uᵢₙₛ(t) when CGM(t) is over a first threshold Th₁ and G*(t) over a second threshold Th₂.

14. Method according to any of claims 10 to 13, wherein the predefined time T₁ is in the range from 30 to 300 min, and the thresholds Th₁ and Th₂ are defined as: CGM(t)≥140 mg/dL and G*(t)≥180 mg/dL

15. Method according to claim 1, wherein the length of the sliding window t_{w} is set to 60 min.

16. Method according to claim 1, wherein the time constant τ which determines the aggressiveness of the filter is set to two times the sampling time of the CGM.

17. Method according to claim 1, wherein the parameter [symbol] is set to 15 g, being the common size of the available commercial glucose supplements.

18. Method according to claim 1, further comprising an optimization step for tunning the parameters k, thᵢₙₛ, thₛₐₜ, k_{resc}, th_{resc} by applying a cost defined as:
$${J}_{s i m} := {J}_{WAIR} + {J}_{C}$$
wherein:
the term J_{WAIR} penalizes the weighted areas of the CGM exceeding certain thresholds gᵤᵤ, gᵤ, gₗ and gₗₗ as follows:
$${J}_{WAIR} = {a}_{uu} \cdot {\int }_{0}^{Tsim} ( {G}_{uu} ( τ ) - {g}_{uu} ) d τ + {a}_{u} \cdot {\int }_{0}^{Tsim} ( {G}_{u} ( τ ) - {g}_{u} ) d τ ++ {a}_{l} \cdot {\int }_{0}^{Tsim} ( {g}_{l} - {G}_{l} ( τ ) ) + {a}_{ll} \cdot {\int }_{0}^{Tsim} ( {g}_{ll} - {G}_{ll} ( τ ) ) d τ ++ {a}_{resc} \cdot {\int }_{0}^{Tsim} ( {G}_{resc} ( τ ) - {g}_{resc} ) d τ$$
wherein Tₛᵢₘ is the simulation length, the parameters aᵤᵤ, aᵤ, aₗ, aₗₗ, and, a_{resc} are non-negative scalars defining the weight of each term, the thresholds are positive scalars defined so that gᵤ<gₗ<gᵤ<gᵤᵤ, signals Gᵤᵤ(t), Gᵤ(t), Gₗ(t), Gₗₗ(t) correspond to the CGM after being saturated to the enclosing thresholds as follows:
$${G}_{u u} ( t ) := \{ \begin{matrix}{g}_{u u} & if CGM ( t ) \leq {g}_{u u} \\ CGM ( t ) & otherwise\end{matrix}$$
$${G}_{u} ( t ) := \{ \begin{matrix}{g}_{u} & if CGM ( t ) \leq {g}_{u} \\ {g}_{uu} & if CGM ( t ) > {g}_{uu} \\ CGM ( t ) & otherwise\end{matrix}$$
$${G}_{l} ( t ) := \{ \begin{matrix}{g}_{ll} & if CGM ( t ) < {g}_{ll} \\ {g}_{l} & if CGM ( t ) \geq {g}_{l} \\ CGM ( t ) & otherwise\end{matrix}$$
$${G}_{l l} ( t ) := \{ \begin{matrix}{g}_{l l} & if CGM ( t ) \geq {g}_{l l} \\ CGM ( t ) & otherwise\end{matrix}$$
and wherein the last addend of J_{WAIR} weights the glucose rebound after rescue carbohydrate suggestion time to better coordinate rescue carbohydrates suggestions and insulin doses, wherein signal G_{resc}(t) represents the value of the CGM that overpasses a certain limit of the value of g_{resc} in the first minutes before the value of C₆ after rescue carbohydrate suggestions, wherein if a meal occurred before C₆, G_{resc}(t) was calculated until mealtime as defined in:
$${G}_{resc} ( t ) = \{ \begin{matrix}CGM ( t ) & \begin{matrix}if ( CGM ( t ) \geq {g}_{r e s c} ) and \\ t ∈ [ {t}_{resc} , min ( {t}_{r e s c} + {C}_{6} , {t}_{meal} ) ]\end{matrix} \\ {g}_{r e s c} & otherwise\end{matrix}$$
where t_{resc} and tₘₑₐₗ denote the rescue carbohydrates and meals times, respectively; and
the second term of the cost function, J_{C}, constrains the shape and magnitude of the control actions as defined below:
$${J}_{C} = {b}_{a c t} \cdot max ( \frac{{n}_{imc \_ act}}{{n}_{m e a l}} - 1 , 0 ) + {b}_{meal \_ resc} \cdot {\sum }_{i = 1}^{{n}_{meal \_ resc}} {meal\_resc}_{i} ++ {b}_{ex \_ resc} \cdot max ( \frac{{\sum }_{i = 1}^{{n}_{ex \_ resc}} {ex\_resc}_{i}}{{C}_{7} {n}_{ex \_ sessions}} - 1 , 0 )$$
where:
the first addend penalizes situations wherein the number of IMC activations in insulin mode (n_imc_act) is larger than the number of meals (n_meal) in the optimization scenario, wherein the parameter b_{act} is a non-negative scalar weight of the addend;
the second addend constrains the total amount of rescues given after meals, wherein the carbohydrate amount given at meal i is defined as meal_resc_i and n__{meal_resc} denotes the number of rescues given after meals, and the parameter b__{meal_resc} is a non-negative scalar weight of the addend; and
the third addend penalizes situations wherein the amount of suggested carbohydrate per exercise event exceeds a certain value of C₇, wherein the carbohydrate amount suggested after exercise i is denoted as ex_resc_i, and n_ex_resc and n_ex_sessions define the number of exercise-related rescues and exercise events, respectively, being the parameter b _{ex_resc} a non-negative scalar weight of the addend.

19. Method according to claim 18, wherein the thresholds gresc, gll, gl, gu and guu are in the range from 40 to 400 mg/dL; C₆ in the calculation of Gresc(t) is in the range from 5 to 300 min; and the tolerable amount of suggested carbohydrate per exercise event in J_{C}, represented by C₇, is between 5 g and 100 g.

20. Add-on module for being incorporated to an artificial pancreas system comprising a calculation unit configured to carry out the steps of any of claims 1 to 19.

21. Artificial pancreas system for performing the method as defined in any of claims 1 to 19, comprising:
a pump (3), for supplying insulin according to a coordinated control action (u_{c});
a continuous glucose monitor (2) for measuring the plasma glucose (G(t)) signal;
a controller for determining the insulin level to be delivered, and
an add-on module comprising a calculation unit configured to carry out the steps of any of claims 1 to 19.

22. Artificial pancreas system according to claim 21, wherein the artificial pancreas system comprises a controller incorporating a method for limitation of insulin-on-board.

23. Computer program adapted for carrying out the steps of the method according to any of claims 1 to 19 by using the calculation unit defined in any of claims 20 to 22.

24. Computer readable storage medium comprising the computer program according to claim 23.
